(19) United States
(12) Patent Application Publication
(10) Pub. No.: US 2013/0164828 A1
(43) Pub. Date: Jun. 27, 2013

(54) OPTICAL TRANSFECTION

(71) Applicants: Kishan Dholakia, St. Andrews (GB); Na Ma, St. Andrews (GB); Praveen Cheriyan Ashok, St. Andrews (GB); David Stevenson, St. Andrews (GB); Francis James Gunn-Moore, St. Andrews (GB)

(72) Inventors: Kishan Dholakia, St. Andrews (GB); Na Ma, St. Andrews (GB); Praveen Cheriyan Ashok, St. Andrews (GB); David Stevenson, St. Andrews (GB); Francis James Gunn-Moore, St. Andrews (GB)

(73) Assignee: University Court of The University of St. Andrews, St. Andrews (GB)

(21) Appl. No.: 13/643,279

(22) PCT Filed: Jun. 10, 2011
(86) PCT No.: PCT/GB11/00881

§ 371 Date: Feb. 18, 2013

(30) Foreign Application Priority Data
Jun. 11, 2010 (GB) 1009800.2

(51) Int. Cl. C12N 15/85

(52) U.S. Cl. CPC C12N 15/85
USPC 435/285.1; 427/555

(57) ABSTRACT

An integrated fibre based device for transfecting material into a cell comprising an optical fiber that has a lens formed at its end for directing light to a surface of the cell, and a channel for delivery of the material for transfection into the cell.

### FIELD OF THE INVENTION

The present invention relates to a fiber based optical transfection system, and method for adapting a fiber for use in such a system.

### BACKGROUND OF THE INVENTION

The introduction of therapeutic and other agents into cells, which are otherwise membrane impermeable, remains a key requirement in cell biology. Currently, a variety of transfection methods are used to solve this problem, including chemical, physical, optical, electrical, and viral. Optical transfection offers selectivity, specificity, high transfection efficiency and good post-transfection cell viability. By applying a tightly focused laser beam on the cell membrane, optical transfection can transiently and locally increase the permeability of the cell's plasma membrane to allow for example nucleic acids to be internalized.

Most optical transfection techniques that have been used employ free space (bulky) optical setups which limit the potential application of the technology for in-vivo experiments. In addition, the transfection efficiency achieved is highly dependent on the quality of the photoporation beam, so expertise in optical alignment is necessary to achieve efficient transfection.

Fiber based femtosecond optical transfection has been proposed. This uses an axicon tipped optical fiber for light delivery. The axicon tip is made using hydrogen fluoride based etching, which makes the fabrication hazardous. Also the transfection efficiency is very sensitive to the quality of the axicon tip. In addition, the short working distance produced by the axicon makes the targeting of the beam focus at the cell membrane very difficult: particular care has to be taken to make sure both fiber tip and cells are not damaged.

Microlensed fibers are widely used in the field of communication for increasing coupling efficiency between terminals and interconnect. Various fabrication procedures are reported for the fabrication of microlensed fibers. Melting the fiber tip by an electric arc discharge or heating to form a lens are the most widely used methods to fabricate a communication standard microlensed fiber. However, these methods do not provide high reproducibility and only lenses with a comparatively large radius of curvature can be fabricated. Polishing can be used to make axicon lenses of different angles; however, this is complex, time consuming and expensive. Femtosecond two-photon lithography is a highly flexible technique, in which micro-structures are directly inscribed on surfaces point by point. However, this technology is in its infancy and the manufacturing cost is unacceptably high for practical applications. Other indirect fabrication methods use coreless silica fiber, micro-silica spheres or a combination of these. All these procedures have disadvantages such as complexity, high cost or lack of flexibility.

### SUMMARY OF THE INVENTION

According to the present invention there is provided a system for delivery of a material, for example a drug or DNA, into a cell, the system comprising an optical fiber and a lens formed on the end of the optical fiber. The optical fiber may be a single mode fiber. The system includes a delivery tube or channel for localized delivery of the material to the cell. Preferably, the optical fiber is positioned in the tube or channel. The delivery tube or channel may be a microcapilliary.

The system may include a laser, for example at least one of: femtosecond laser, nano-second laser, pico-second laser and continuous wave laser. The laser is provided for optically porating a sample, for example a cell.

The system may include a multimode fiber for delivering an illumination beam for illuminating a sample area, so that the sample being treated can be viewed during and/or after treatment.

According to another aspect of the invention there is provided a method for fabricating a microlens on a fiber using an optically curable material, for example an ultraviolet (UV) curable adhesive. The lens characteristics can be tailored by changing the parameters of curing the adhesive. Using this technique microlenses yielding a very small focal spot (2-3 μm) at a relatively large working distance (−15 μm) can be made.

The method of the invention involves applying an optically curable material to an end of the fiber and exposing the end of the fiber to a laser radiation suitable for curing the material. Depending on the profile of the curing beam, different types of micro-lenses having potentially different applications can be made. The fabrication procedure is simple and requires only basic focusing optics, and relatively simple, low power lasers, for example a blue diode laser, to achieve preferential and controlled curing of the optical curable material. The beam shape of the curing laser beam may be modified to obtain a wide variety of microlenses with different shapes and parameters.

The method may involve removing uncured material remaining after exposure to the curing radiation. The method may also involve varying curing exposure time, curing exposure rate, alignment of the fiber tip with respect to the curing beam for obtaining different types of microlens.

The method may involve forming a drop of the liquid form of uncured, but optically curable, material on the end of the fiber. The method may involve dipping the end of the fiber into the optically curable material so that some of the optically curable material adheres to the end of the fiber.

The optically curable material may be sensitive to UV radiation and/or may be an adhesive.

The microlens may be shaped to tightly focus, collimate or cause divergence of the output from the optical fiber.

### BRIEF DESCRIPTION OF THE DRAWINGS

Various aspects of the present invention will now be described by way of example only and with reference to the accompanying drawings, of which:

FIG. 1(a) is an image of an integrated fiber based optical transfection device;

FIG. 1(b) is an expanded view of part of the device of FIG. 1(a);

FIG. 1(c) is another expanded view of a part of the device of FIG. 1(a);

FIG. 2 is a schematic view of a fiber based optical transfection system that includes the fiber based optical transfection device of FIG. 1;

FIG. 3 shows images of cells recorded using the system of FIG. 2;

FIG. 4 is an image of successfully transfected cells (expressing a red fluorescent protein);

FIG. 5 is a plot showing transfection efficiency of CHO-K1 cells achieved using an axicon tipped fiber, a microlens tipped fiber and the system of FIG. 2, respectively, as well as transfection efficiency of HEK-293, also measured using the system of FIG. 2;

FIG. 6 is a schematic view of a system for fabricating a microlens;

FIG. 7 illustrates various steps in a method for making microlens tipped fiber;

FIG. 8 is an SEM image of a microlens formed using the method of FIG. 7;

FIG. 9 shows the results of a ray trace model for a lens made using the method of FIG. 7, and

FIG. 10 shows examples of different structures that can be made by changing the curing beam distribution in the method of FIG. 7.

### DETAILED DESCRIPTION OF THE DRAWINGS

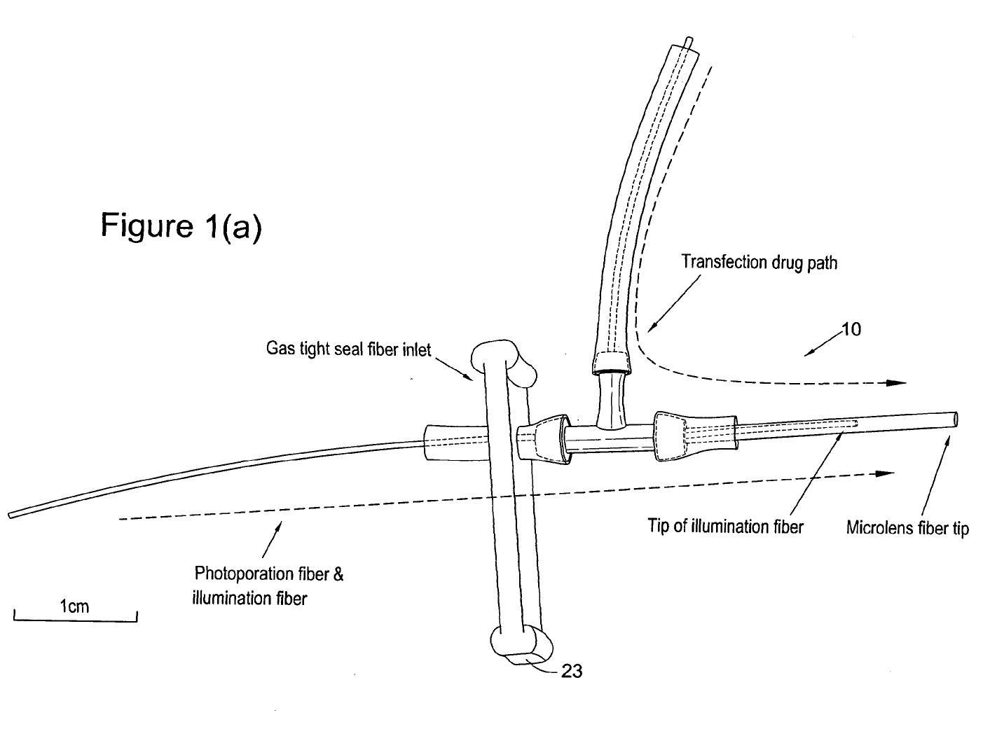

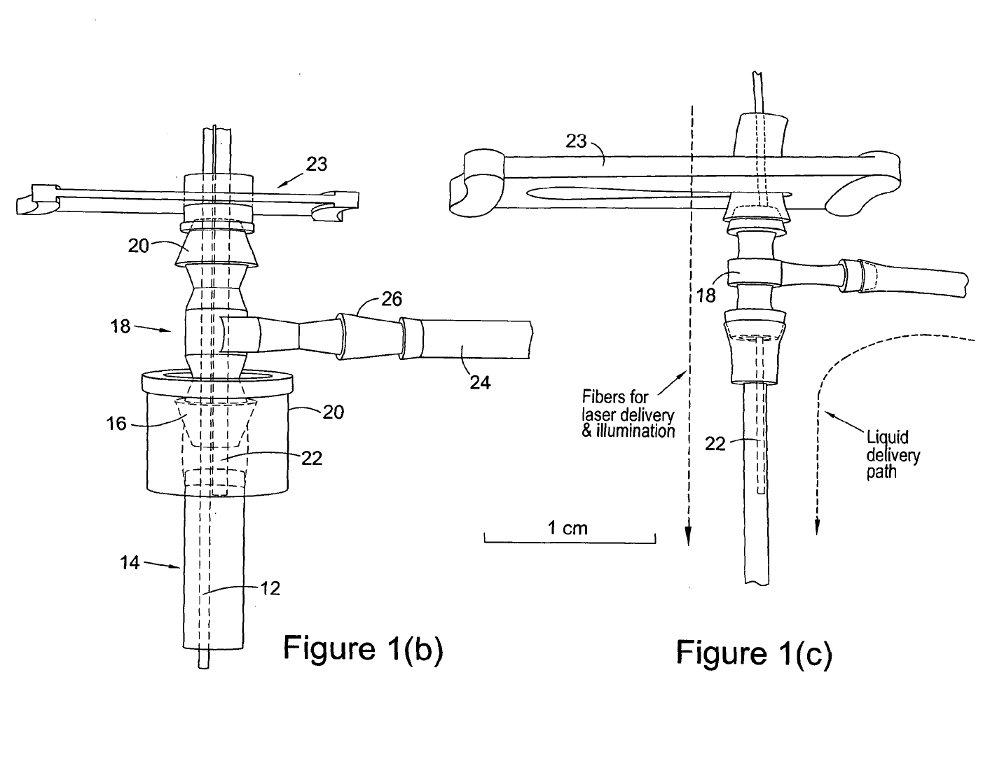

FIGS. 1(a) to (c) show an intergrated fiber based optical transfection device 10. This has a fiber with a lens formed at its tip (microlensed fiber) 12 for delivering a focused laser beam near a cell membrane. A technique for forming the lens on the end of the fiber will be described in more detail later. To achieve localized drug delivery during optical transfection, the microlensed fiber 12 is integrated within a capillary tube 14, which is used to deliver fluid containing the material that is to be transfected to the cell. The micro-capillary 14 is attached to a first port 16 of a barbed three port T connector 18 (Harvard Apparatus, 72-1487) using flexible plastic tubing 20 (for example Tygon T3601, inner diameter=0.8 mm, outer diameter=2.4 mm). The microlensed fiber 12 is inserted through the second port 20, into the micro-capillary 14 (inner diameter=0.58 mm, outer diameter=1 mm) and the tip of the fiber 12 positioned close to the tip of the micro-capillary 14. A cleaved multimode fiber 22 of core diameter 200 μm (Thorlabs, BFL37-200) is inserted into the micro-capillary 14 for illuminating the sample. The optimum distance of the tip of the multimode fiber 22 from the apex of the microlens was experimentally estimated to be ˜1 cm from the tip of microlens tipped fiber to achieve the best contrast for the sample. A slide clamp 23 (WPI, Luer Valve Assortment, 14042) was used to seal the flexible tubing, attached to the fiber inlet port, in order to ensure that the device 10 was airtight during sample injection. Flexible tubing 24 is attached to the third port 26 for delivery of the material to be transfected, for example DNA.

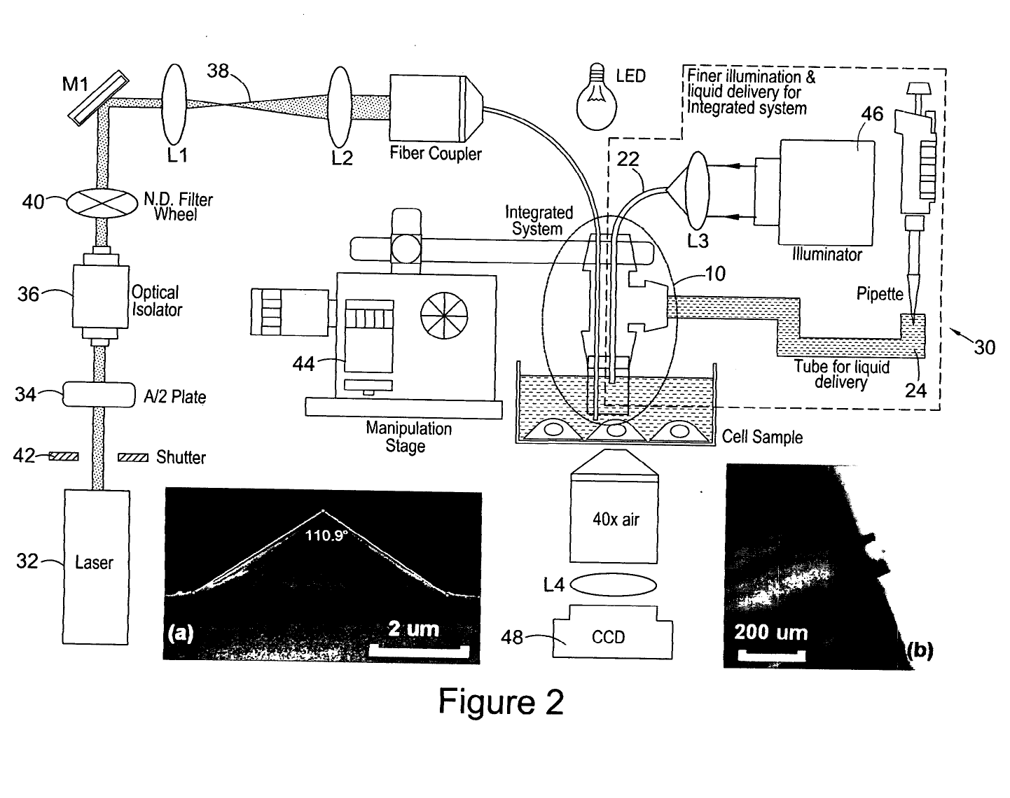

FIG. 2 shows a photoporation system 30 that can be used with the integrated device 10 of FIG. 1. A laser 32, for example a Ti: Sapphire laser, is used to form a collimated poration laser beam, which is directed into a combination of a half wave plate 34 and an optical isolator 36 (Laser2000, UK, I-80-2) to eliminate back reflection from the beam path. A magnifying telescope 38 (typical magnification 1.6×) is used to expand the incoming laser beam, which is coupled to the microlensed optical fiber 12 through a fiber collimator (Thorlabs, F810FC-780). The fiber output power is adjusted using a variable neutral density (ND) filter wheel 40 appropriately placed in the beam path. During photoporation collimated light from the laser 32 is focused by the microlensed fiber 12 onto the cell membrane. The average power of the beam is kept at 20 mW, with a peak power per pulse of 0.24 kW. A mechanical shutter 42 is used to control the time duration of the laser dosage on the cell membrane. The integrated transfection device 10 used for poration is mounted on a three axis (X-Y-Z) translation stage 44 for insertion into the sample medium. An illuminator 46 is provided for illuminating the sample using the illumination fiber. Liquid containing the material that is to be transfected is delivered using the liquid delivery tube 24 connected to the micro-capillary 14. Below the sample area is provided a camera 48 for capturing images of the cells before, during and after transfection.

A sample including cells that were to be porated was put in a petri dish. To transfect material into the sample cells, poration was instigated by the laser emitting at 800 nm, with output pulse duration of ˜100 fs and a pulse repetition frequency of 80 MHz (Coherent, MIRA). For the purposes of comparison, this was done for both an axicon tipped fiber (as known in the prior art) and a microlens tipped fiber. At the output end of both the microlens and axicon tipped fibers, the pulses undergo stretching due to a non-linear phenomena occurring inside the fiber—self-phase modulation (SPM) and group velocity dispersion (GVD)—giving an overall pulse duration of approximately 800 fs. Axicon tipped fiber transfection was performed as described previously. For microlens tipped fiber transfection, due to restrictions imposed by the geometry of the fiber and the imaging path, the fiber was tilted at ˜5-10° with respect to the vertical axis. With a white LED light source on top and an imaging system below the sample, the sample cells were observed during the transfection procedure using the camera below the sample.

Two cell samples were tested in the system of FIG. 2. These were CHO-K1 and HEK-293. The cells were cultured in modified eagles medium (MEM) containing 10% foetal calf serum (FCS), 18 IU/ml of penicillin, 18 μg/ml of streptomycin, 1.8 mM of L-Glutamine (“complete medium”) in a humidified atmosphere of 5% CO2/95% air at 37° C. Cells were grown to sub-confluence in 30 mm diameter glass-bottomed culture dishes (World Precision Instruments, Stevenage, UK) in 2 ml of culturing cell media (MEM). Prior to experimentation, the cell monolayer was washed twice with OptiMEM (Invitrogen) and for all experiments (except the integrated system where the solution was delivered microfluidically), the sample was bathed in 1 ml solution of OptiMEM containing 9 μg/ml mitoDsRED plasmid, encoding a mitochondrially targeted Discoideum Red Fluorescent protein (BD Biosciences, Oxford, UK).

To ensure the sterility of the drug delivery system, before each transfection experiment 2 ml of 70% ethanol was run through to sterilize the whole system and was subsequently dried using filtered air. The capillary tube was tested for multiple transfection experiments and the cell viability for subsequent experiments showed that the system remains sterile with the above mentioned sterilization procedure.

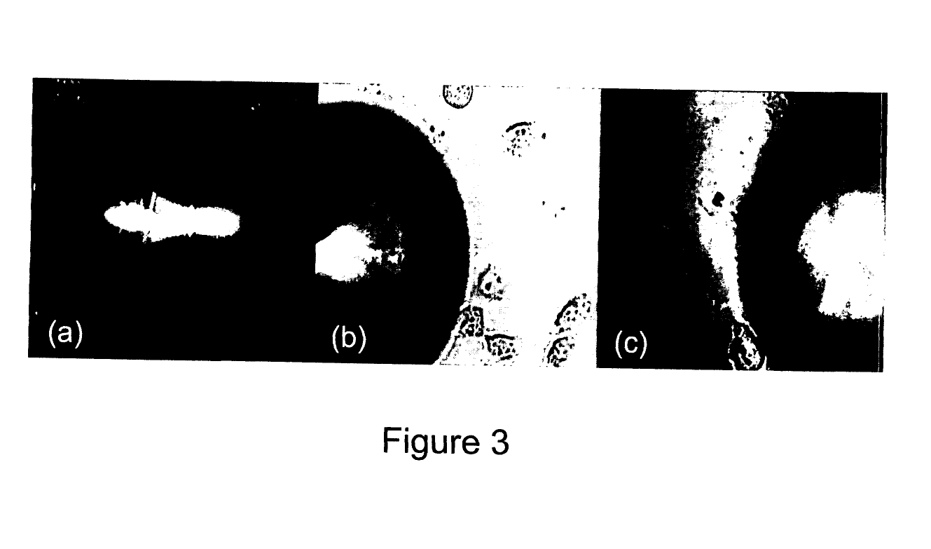

The pipette loaded with sample was connected to the capillary tube of the integrated system. Controlled injection of DNA locally into CHO-K1 and HEK-293 was achieved using the pipette during optical transfection. An image of cells recorded during optical transfection with the integrated illumination system, is shown in FIG. 3. Despite the poor image contrast due to a shadow cast by the photoporation fiber, when targeted cells were imaged the cell boundaries were visible, which permitted them to be transfected.

In order to monitor potentially spontaneous transfected cells, each photoporated sample dish was accompanied by a control sample dish in which cells were cultured, bathed in plasmid DNA solution and then experienced the fiber presence in the absence of laser radiation. Experimental details of the number of treated cells and the results are shown in Table. 1. The number of spontaneously transfected cells varied between 0-2 cells for each sample dish.

TABLE 1

| | | | | Transfection |
|---|---|---|---|---|
| | | No of Dish | Total No. of | Efficiency |
| | Cell type | treated | treated cells | (±SEM) (%) |
| Axicon | CHO-K1 | 15 | 450 | 30.22 ± 5.36 |
| tipped fiber | | | | |
| Microlens | CHO-K1 | 20 | 800 | 40.25 ± 3.39 |
| tipped fiber | | | | |
| Integrated | CHO-K1 | 15 | 525 | 45.71 ± 4.84 |
| system | HEK-293 | 5 | 175 | 64.00 ± 4.10 |

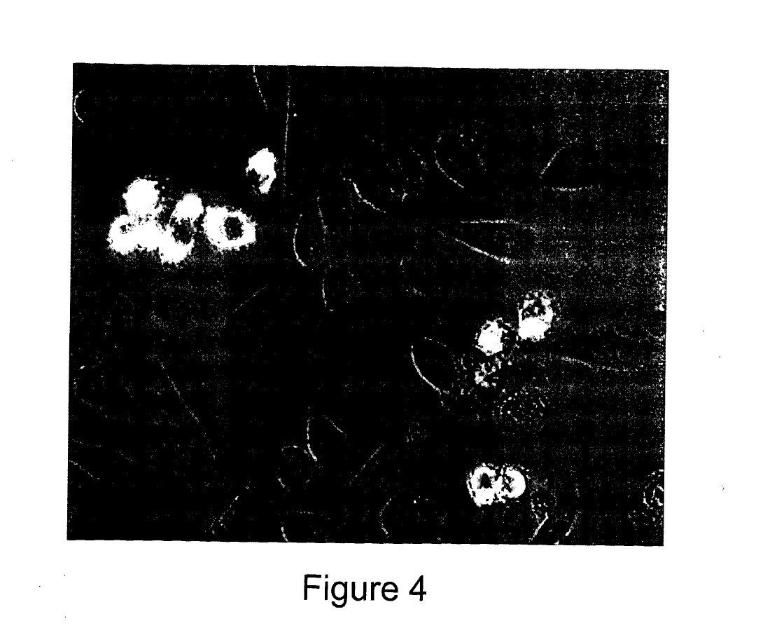

During laser irradiation no visual response was observed. After the laser treatment, the cell monolayer was bathed in complete medium and returned to the incubator. The sample was viewed forty eight hours later under a fluorescent microscope, where successfully transfected cells expressed the red fluorescent protein as shown in FIG. 4.

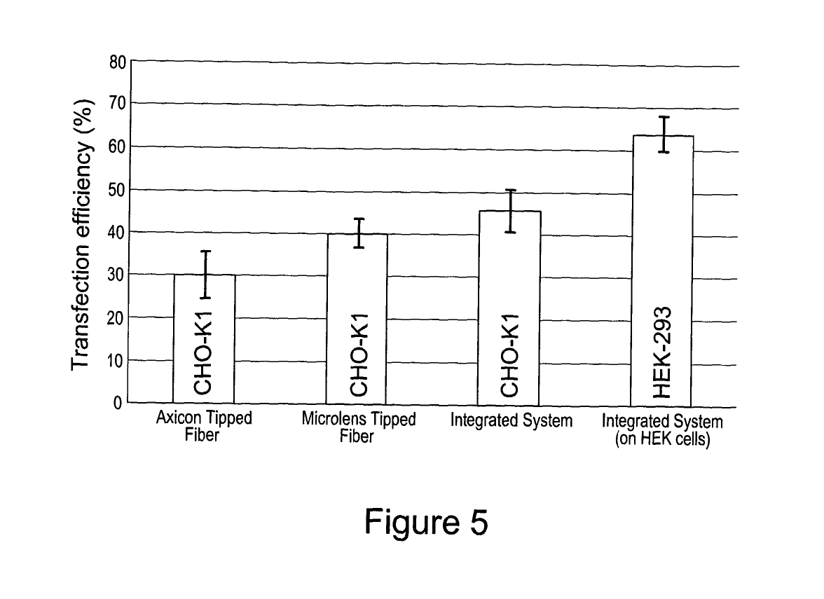

FIG. 5 shows a comparison of the transfection efficiency of CHO-K1 cells achieved using an axicon tipped fiber, a simple microlens tipped fiber (no localized fluid delivery) and the integrated device 10 of FIGS. 1 and 2 respectively. In addition, FIG. 5 shows the transfection efficiency of HEK-293 when treated with the integrated device 10. The transfection efficiency is defined as the number of cells expressing the correctly targeted red fluorescent protein 48 hours after laser treatment divided by the total number of cells that were laser treated in a particular area of interest.

From FIG. 5, it can be seen that the efficiency of the fiber based optical transfection technique of the present invention is comparable to that of free space transfection. Also, the microlens tipped fiber provides a higher transfection efficiency and smaller standard deviation in efficiency, than the axicon tipped fiber method. This reflects the fact that the longer working distance makes the manipulation of a microlens tipped fiber easier and more stable compared to an axicon tipped fiber. During the transfection procedure, the axial focal position needed to be found only at the beginning of the procedure and then multiple cells in the same sample dish could be photoporated just by moving the fiber mount laterally. This causes less damage to cells and the fiber tip, high transfection efficiency and more consistency.

Using the integrated device 10 of the invention, highly localized delivery of DNA-containing fluid can be achieved. The long working distance lens allows easy manipulation of the fiber tip over the sample and hence better throughput for photoporation compared to its axicon tipped counterpart. The localized drug delivery makes the technology amenable to single cell studies. Cell boundaries can be observed during transfection using a multimode fiber based illumination system embedded into the integrated system. Using microlensed fiber with an integrated delivery channel opens up prospects for a portable “hand-held” system that can locally deliver therapeutic agents and transfect cells within a fiber geometry placing minimal requirements upon any microscope system.

Another aspect of this invention provides a method for making a fiber with a microlens on its tip using a UV curable adhesive. To demonstrate the effectiveness of this technique a lens was fabricated on a commercially available single mode fiber. This has a mode field diameter of 5.6 μm, cladding diameter 125 μm, and an operating wavelength of 830±100 nm (Thorlabs, SM800-5.6-125). A UV curable adhesive (Norland, NOA 65) with optimum sensitivity for curing in the 350-380 nm range was used due to its good adhesion to glass, fast curing time, easy processing, suitable refractive index (1.524 for polymerized resin) and high transmission efficiency (˜98%) at 800 nm. These characteristics make the polymer lens ideal for the delivery of high peak power pulsed laser light without damaging the structure. The UV curable adhesive was cured with a laser beam specially shaped to obtain a desired shape of the lens.

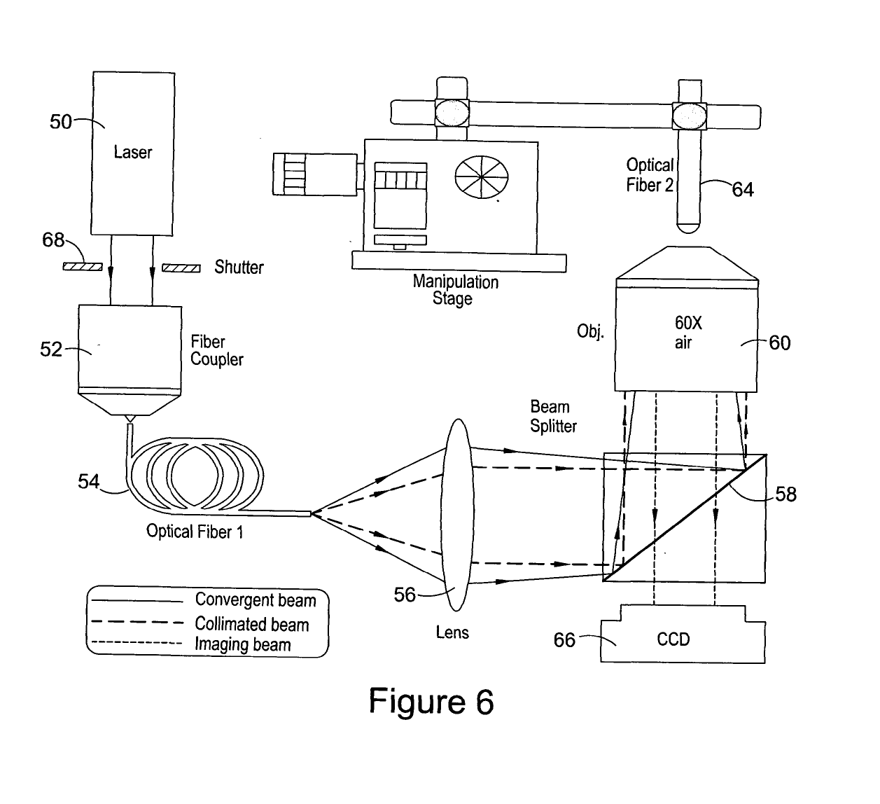

FIG. 6 shows a set up for fabricating a microlens using free space optics for directing a laser beam onto the end of an optical fiber. This has a violet diode laser 50 (405 nm) coupled through an objective 52 (×10, Newport, UK) into a single mode optical fiber 54 (Thorlabs, S405-HP) with a coupling efficiency of ˜45%, in order to improve the lateral profile of the laser beam. The end of the fiber is positioned at the focal point of a lens 56, which collimates light from the laser and directs it onto a beam splitter 58. The lateral beam profile of the output beam is measured using a long working distance objective (Mitutoyo ×100 infinity-corrected, WD=6 mm) to confirm a high quality TEM₀₀ Gaussian beam profile. The laser beam is then directed to an objective 60 (×60 Nikon) which focuses the light to a fabrication area, where a fiber 62 that is to be processed is vertically mounted on a xyz translation stage 64. A CCD camera (WAT-250D) is provided to allow the tip of the fiber to be imaged during the curing process. Exposure time is controlled using a shutter 68 (Newport, UK, model 845HP-02) at the output of the laser 50.

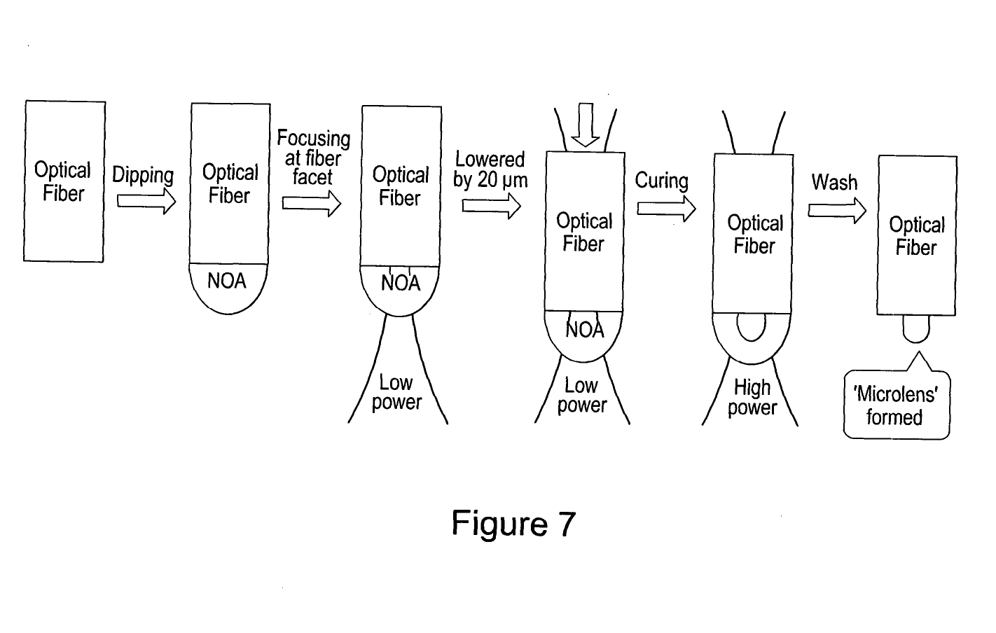

FIG. 7 shows the steps involved in microlens fabrication. A well-cleaved optical fiber is vertically dipped and raised from a drop of UV curable adhesive such that a hemisphere of adhesive on the fiber tip is formed. The fiber is then mounted to the curing setup of FIG. 6. Keeping the power of the laser lower than the threshold (<0.1 mW), below which curing process would not be initiated, the laser beam was positioned at the center of fiber tip facet and defocused by 20 μm from the tip in order to get correct beam shape to produce the desired lens structure. In the example shown, the beam has a Gaussian profile, and the fiber and the beam are positioned, so that the beam waist is located in the region of the fiber tip. The power of the laser is increased to 0.5 mW to start the curing process. At the beginning of the polymerization process, the UV adhesive partially cures around the center of fiber facet followed by a growth towards the direction of the laser. After exposure for 5 s, the un-polymerized adhesive was removed using acetone and a ‘micro-stick’ is created. At the apex of ‘micro-stick’ a curved facet is formed which acts as a focusing surface for the output beam from the fiber.

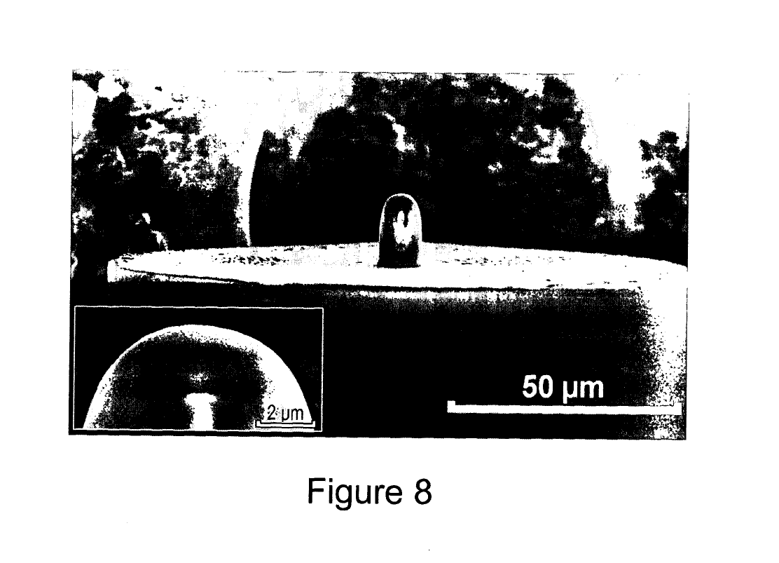

FIG. 8 shows an SEM image of a microlens formed using this technique. The physical parameters of the lens were estimated from the SEM images. The height of the lens was estimated as 11±1 μm, base diameter 7±1 μm and top diameter 5±1 μm. An 800 nm laser from a Ti-Sapphire laser (Coherent, MIRA) was coupled to the microlens tipped fiber and the output beam from microlens was profiled in water from a series of lateral cross-sections with a 5 μm step change using a water immersion objective (×60 Olympus UPlanSApo) in conjunction with a CCD camera (WAT-250D). The working distance of the lens (distance of the focal plane from the apex of the lens) and the diameter of the focal spot were estimated from the beam. The estimated working distance of the beam was 15±5 μm, focal spot diameter was 3±0.05 μm and the beam divergence was 15°.

A ray tracing model of the lens fabricated at the tip of the fiber was built using optical design software (Zemax Development Corporation) from the parameters estimated from the SEM images and beam profiling. In the Zemax model, a radial source with a Gaussian profile was defined at a wavelength of 800 nm, which propagates from a cylinder of refractive index similar to that of the core of the fiber used. The output beam from the cylinder had same numerical aperture (NA=0.12) and mode field diameter (MFD=5.6 μm), as was defined by the specifications of the single mode fiber used for the experiments. A microlens was defined at the surface of the cylinder with a material of refractive index same as that of cured UV adhesive (Norland 65—refractive index ˜1.52). The lens was designed with physical dimensions estimated from the SEM image, keeping the radius of curvature of the surface close to the surface of the cylinder as 0 and the radius of curvature of the second surface (apex of the microlens) as a variable. The whole system was immersed in water and the radius of curvature of the lens was estimated which provided the experimentally measured working distance.

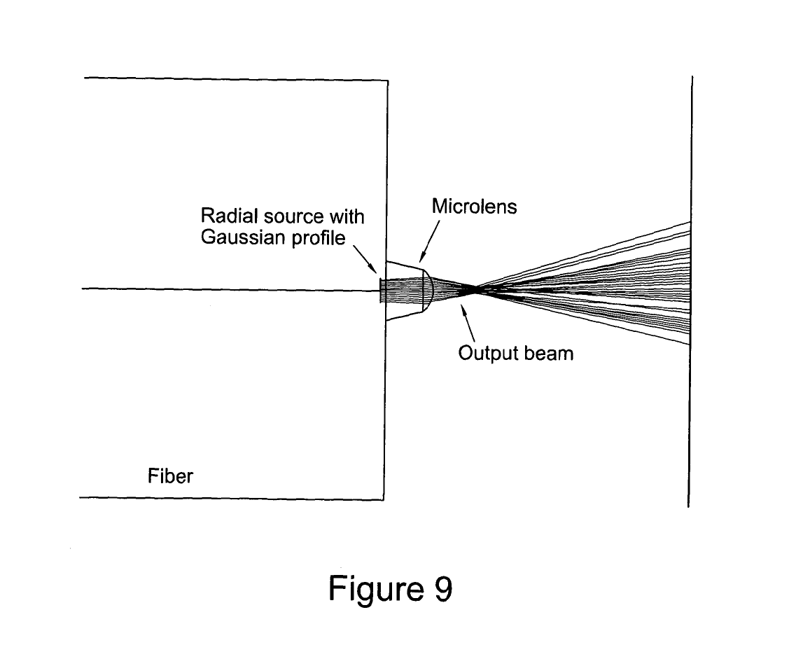

FIG. 9 shows the results of the ray trace model. From this, it can be seen that the radius of curvature was estimated to be 7 μm±0.5 μm. With all the estimated parameters, beam profiling was performed on the model at a step size change of 5 μm and the diameter of the focal spot (2.9 μm) and divergence (16°) were calculated. These were found to be comparable with the experimental values.

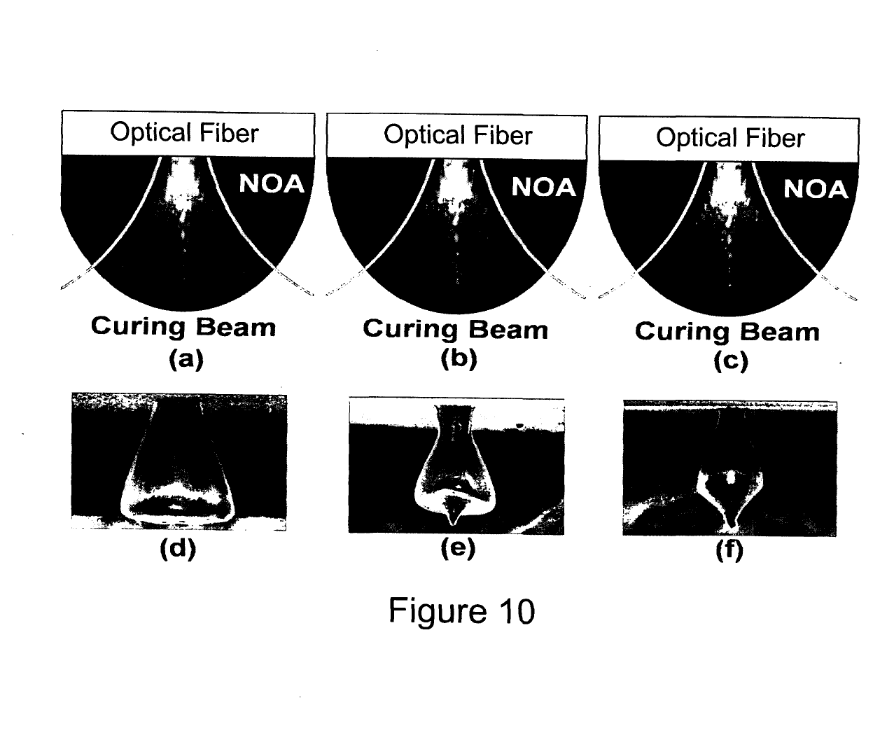

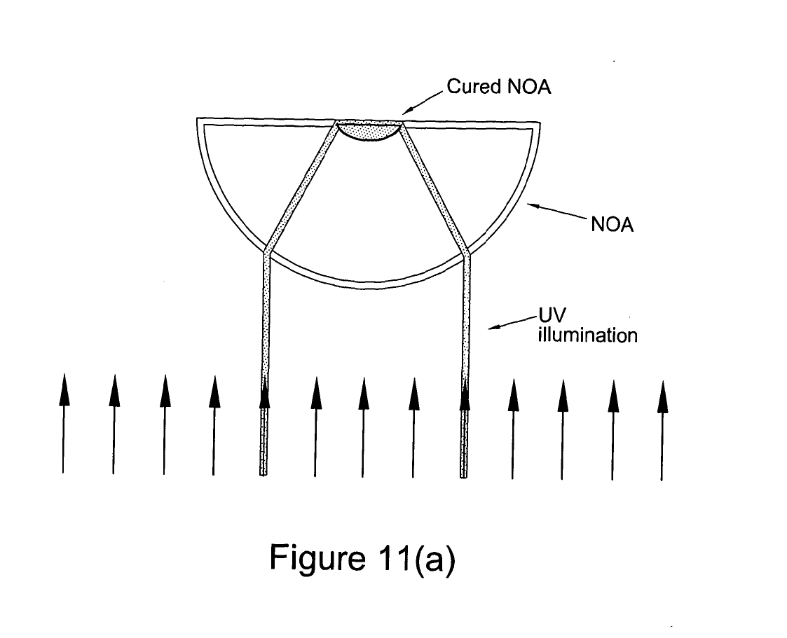

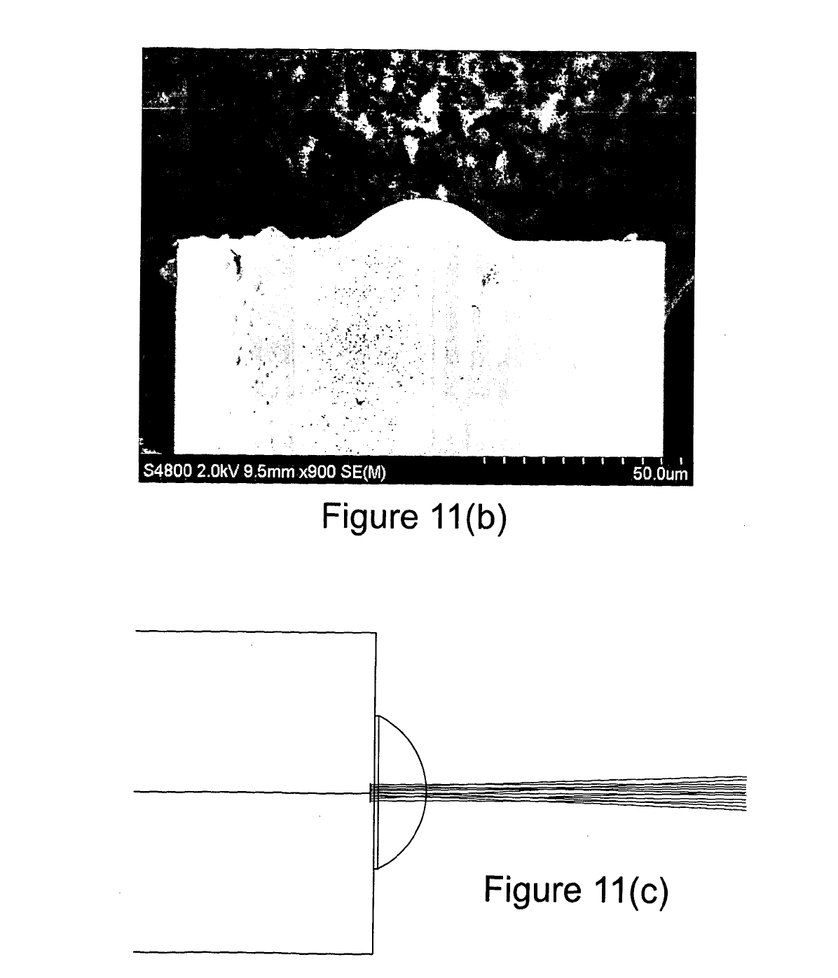

The UV curing fabrication procedure is highly flexible. By changing parameters such the light distribution near to the focus of the curing beam, intensity of the curing beam or curing time, it is possible to fabricate different structures at the tip of the fiber. FIG. 10 shows examples of different structures that can be made by changing the curing beam distribution. Using curing beam (a), (b) and (c) with appropriate laser power and curing time structures like (d), (e) and (f) respectively were fabricated. As shown in FIG. 10, the structures created are strongly related to the beam distribution.

Whilst the description has focused on a fiber lens fabricated using a beam with a Gaussian profile, other curing beam profiles can be used. FIG. 11(a) shows an example of a collimated curing beam. In this case, the collimated beam is directed onto the hemispherical drop of curable material and focused towards the tip of the fiber. This causes a smoothly curved lens to form. This fabrication does not require any intricate alignment of the curing beam. To test this, technique a piece of well cleaved single mode commercially available optical fiber was dipped into UV glue (e.g. Norland optical adhesive) vertically and pulled out slowly. The surface tension of UV glue allowed a hemisphere to be formed on the end of fiber tip. Then a collimated mercury lamp illumination was used to cure UV glue hemisphere. Due to the self-focusing property of hemisphere, the curing beam focused around the center of the end of fiber tip, and so polymerization started from there and grew outward. The curing power and time determined the size of microlens and so has to be controlled depending on the size requirements. Once curing was complete, acetone was used to wash off un-polymerized UV glue. FIG. 11(b) shows an image of a lens formed using this technique. FIG. 11(c) shows the corresponding ray diagram, which illustrates the quasi-collimated output. Lenses of this type can be used in micro-endoscopic devices for bio-imaging, and in communication to enhance the coupling efficiency.

The microlens tipped fiber and the transfection system of the present invention provide numerous advantages over prior art systems. For example, the microlens tipped fiber can be designed to have a longer working distance (15-20 μm) compared to the axicon tipped fiber of the prior art, which makes it easy to position and focus on a cell membrane. In contrast to the axicon tipped fiber based transfection, transfection with a microlensed fiber does not need focusing and re-focusing for transfection of each cell. During the transfection experiments described above, the beam focus was fixed at 5 pm above bottom of the sample petri dish, which was the average height of the cells being investigated. Without any further axial positioning, the tip of the microlensed fiber could be laterally scanned in order to transfect different, individual cells within one Petri dish.

A skilled person will appreciate that variations of the disclosed arrangements are possible without departing from the invention. Whilst the transfection of DNA is described above, it will be appreciated that any material of interest could be introduced into the cell, for example RNA, and various dyes such as Propedium Iodide (PI) and Tryphan Blue. Accordingly the above description of the specific embodiment is made by way of example only and not for the purposes of limitation. It will be clear to the skilled person that minor modifications may be made without significant changes to the operation described.

1. A system for transfecting material into a cell comprising an optical fiber that has a lens formed at its end for directing light to a surface of the cell, and a delivery channel for delivery of the material for transfection into the cell.

2. A system as claimed in claim 1 comprising at least one laser with an output coupled into the optical fiber for delivery of light to the surface of the cell, thereby to form an opening in the cell to allow material to be transfected.

3. A system as claimed in claim 2 wherein the at least one laser is selected from: a femtosecond laser, nano-second laser, pico-second laser and continuous wave laser.

4. A system as claimed in claim 1 wherein the optical fiber is in the delivery channel.

5. A system as claimed in claim 1 wherein the delivery channel comprises a microcapilliary.

6. A system as claimed in claim 1 comprising illuminating means for illuminating the cell.

7. A system as claimed in claim 1, wherein the lens at the end of the fiber is a focusing lens.

8. A method for forming a lens on an end of an optical fiber comprising applying an optically curable material to an end of the fiber and exposing the end of the fiber to a curing laser beam suitable for curing the material, wherein the shape of the lens is defined by the profile of the curing beam.

9. A method as claimed in claim 8 comprising removing uncured material remaining after exposure to the curing beam.

10. A method as claimed in claim 8 wherein the laser beam is provided externally of the fiber.

11. A method as claimed in claim 8 comprising varying the profile of the curing beam, to modify the shape of the lens formed.

12. A method as claimed in claim 8 comprising selecting or varying one or more parameters of the curing beam to define the shape of the lens formed.

13. A method as claimed in claim 12 wherein the parameters of the curing beam comprise one or more of: curing exposure time, power of the curing beam, alignment of the fiber tip with respect to the curing beam.

14. A method as claimed in claim 8 wherein the curing beam has a Guassian profile.

15. A method as claimed in claim 8 wherein the curing beam is such that a focusing lens is formed at the end of the fiber.

16. A method as claimed in claim 8 comprising forming a drop of the optically curable material on the end of the fiber.

17. A method as claimed in claim 16 comprising dipping the end of the fiber into the optically curable material so that some of the optically curable material adheres to the end of the fiber.

18. A method as claimed in claim 8 wherein the optically curable material is an adhesive and/or is sensitive to UV radiation.

19. A system as claimed in claim 1 comprising an optical fiber with a lens at its end made by forming a lens on an end of an optical fiber comprising applying an optically curable material to an end of the fiber and exposing the end of the fiber to a curing laser beam suitable for curing the material, wherein the shape of the lens is defined by the profile of the curing beam.

20. An integrated device for transfecting material into a cell comprising an optical fiber that has a lens formed at its end for directing light to a surface of the cell, and a delivery channel for delivery of the material for transfection into the cell.

21. An integrated device as claimed in claim 20 wherein an inlet port is provided to allow connection of a fluid delivery supply to the integrated delivery channel.
